(19) United States
(12) Patent Application Publication
(10) Pub. No.: US 2008/0181932 A1
(43) Pub. Date: Jul. 31, 2008

(54) COMPOSITIONS FOR ORAL DELIVERY OF PHARMACEUTICALS

(75) Inventors: Jonathan David Bortz, Saint Louis, MO (US); Paul Timothy Brady, Saint Louis, MO (US); Yury Lagoviyer, Olivette, MO (US)

Correspondence Address: KV PHARMACEUTICAL COMPANY, 4080B WEDGEWAY COURT, EARTH CITY, MO, 63045, US

(73) Assignee: DRUGTECH CORPORATION, Wilmington, DE (US)

(21) Appl. No.: 12/021,438

(22) Filed: Jan. 29, 2008

Related U.S. Application Data
(60) Provisional application No. 60/887,227, filed on Jan. 30, 2007.

(51) Int. Cl. A61K 9/50; A61K 38/43; A61K 9/52; A61K 9/00; A61P 43/00

(52) U.S. Cl. 424/439; 424/457; 424/94.1; 424/452

(57) ABSTRACT

The invention provides methods and compositions for the oral delivery of pharmaceutically active agents. In particular, the compositions generally comprise a plurality of pharmaceutically active agents embedded in a matrix that is substantially erodable when contacted with an aqueous medium. The compositions may also include an inner core comprising an inert material. The compositions may be introduced into the oral cavity of a subject by liquid beverage or food product comprising a composition of the invention.

This application claims the benefit of U.S. provisional patent application Ser. No. 60/887,227, filed on Jan. 30, 2007, the entire disclosure of which is incorporated by reference herein.

### FIELD OF THE INVENTION

The present invention relates to compositions for the oral delivery of pharmaceutically active agents. In particular, the compositions generally comprise a plurality of pharmaceutically active agents embedded in a matrix that is substantially erodable when contacted with an aqueous medium. The compositions may also include an inner core comprising an inert material.

### BACKGROUND OF THE INVENTION

For optimal pharmacotherapy, it is important to achieve maximum patient compliance. Compliance is dependent on a number of factors including but not limited to the route and frequency of drug administration. Frequency of administration can sometimes be decreased by administering long-acting, sustained release, or controlled release pharmaceutical formulations. These techniques have been of tremendous benefit, especially for oral administration. But oral dosage forms themselves oftentimes have serious disadvantages that adversely affect patient compliance.

Oral dosage forms present significant drawbacks for several classes of patients. Many patients are unable or unwilling to swallow a solid dosage form. This problem occurs primarily in children and the elderly, however, problems with swallowing are not limited to those segments of the population. Certain conditions or disease states manifest themselves by swallowing difficulties. Otherwise healthy individuals can also exhibit problems with swallowing. Such swallowing difficulties, irrespective of their cause, can severely compromise patient compliance. Swallowing difficulties are also problematic when medicating animals.

While the pharmaceutical industry has long-recognized the need for a satisfactory alternative to oral dosage forms, none have materialized. Syrups, elixirs, microcapsules containing slurries, chewable tablets and other novel tablet or capsule dosage forms have been developed. None of these dosage forms have been ideal and each has their own disadvantages. To improve patient compliance, therefore, a need remains for an oral delivery composition that is easy to swallow.

### SUMMARY OF THE INVENTION

One aspect of the invention encompasses a composition comprising an outer layer formed over an inner core. The inner core typically comprises an inert material and the outer layer comprises a plurality of pharmaceutically active agents embedded in a matrix that is substantially erodable when contacted with an aqueous medium.

Another aspect of the invention provides a composition comprising an outer layer formed over an inner core. The inner core typically comprises an inert material and the outer layer comprises a plurality of microcapsules embedded in a matrix that is substantially erodable when contacted with an aqueous medium. The microcapsules comprise a pharmaceutically active agent and a coating that encapsulates the pharmaceutically active agent.

Yet another aspect of the invention encompasses a composition comprising a plurality of microcapsules embedded in a matrix that is substantially erodable when contacted with an aqueous medium. The microcapsules comprise a pharmaceutically active agent and a coating that encapsulates the pharmaceutically active agent. Additionally, the microcapsules have an average diameter of less than approximately 100 microns.

A further aspect of the invention provides a method for orally delivering a pharmaceutically active agent to a subject. The method comprises introducing an oral delivery composition of the invention into the oral cavity of the subject.

Other iterations of the invention are described in more detail herein.

### FIGURES

FIG. 1 is a schematic depicting an embodiment of an oral delivery composition with no inner core.

FIG. 2 is a schematic depicting an embodiment of an oral delivery composition having an inner core.

FIG. 3 is a schematic depicting an embodiment of an oral delivery composition having an inner core with an indicator coating.

### DETAILED DESCRIPTION OF THE INVENTION

The present invention provides compositions for the oral delivery of pharmaceutically active agents. Typically, the composition comprises a plurality of pharmaceutically active agents embedded in a matrix that is substantially erodable when contacted with an aqueous medium. As such, when the compositions are contacted with saliva or water the matrix erodes and releases the pharmaceutically active agents. The pharmaceutically active agents are typically coated so that if they are released in the mouth of a subject no negative taste occurs. Because the pharmaceutically active agents utilized in the invention are generally less than about 200 microns, the oral delivery compositions can readily be combined with a beverage, such as water or juice, and utilized to administer drugs, vitamins, or minerals to subjects that have difficulty swallowing capsules or tablets, such as pediatric or elderly subjects.

### (I) Composition for Oral Delivery

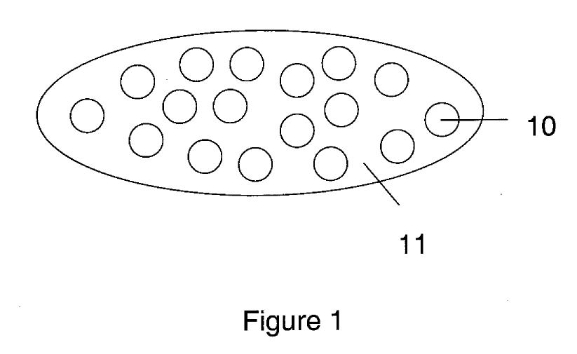

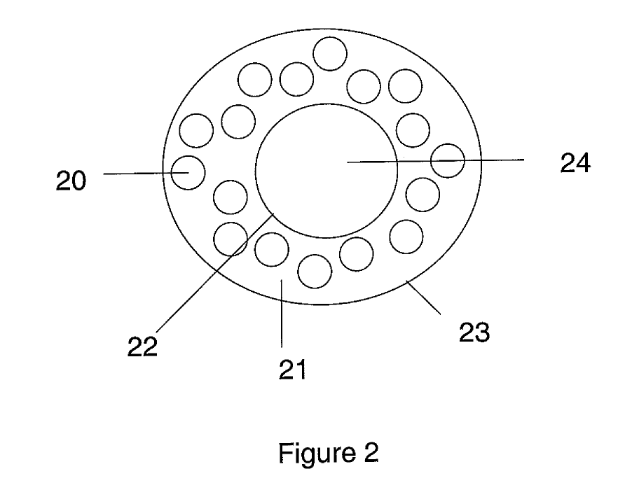

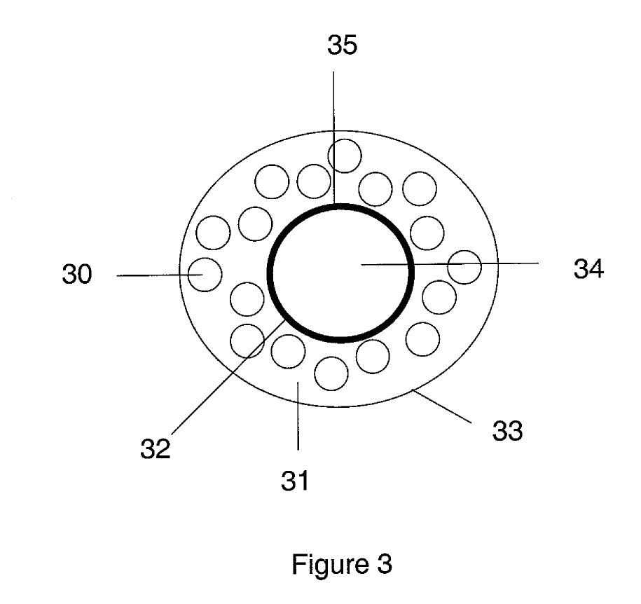

One aspect of the invention encompasses a composition comprising an outer layer formed over an inner core. The outer layer has a plurality of pharmaceutically active agents embedded in a matrix that is substantially erodable when contacted with an aqueous medium. Alternatively, the pharmaceutically active agents may be encapsulated by a coating. The inner core generally comprises an inert material. A representative composition in accordance with this aspect of the invention may be found in FIG. 2 depicting an outer layer 23, having a plurality of pharmaceutically active agents 20, embedded in a matrix 21, the outer layer being formed over the inner core 22, which typically comprises an inert material 24. In certain embodiments, as shown in FIG. 3, an indicator coating 35 may be formed over the inner core 32, which typically comprises an inert material 34, and disposed between the inner core 32 and the outer layer 33. Further, FIG. 3 depicts the outer layer 33 having a plurality of pharmaceutically active agents 30 embedded in a matrix 31. In an alternative embodiment, the composition may comprise an outer layer with no inner core. In this context, represented by FIG. 1, the composition generally comprises a plurality of pharmaceutically active agents 10 embedded in a matrix 11 that is substantially erodable when contacted with an aqueous medium. In certain alternatives of this embodiment, the pharmaceutically active agents may be encapsulated. The size and shape of the oral delivery compositions can and will vary. They may be regularly shaped, irregularly shaped, round, spherical, and combinations thereof. The average diameter of the composition may be on a nanoscale, microscale, or macroscale, which will vary depending upon the embodiment. Each feature of the composition is described in more detail below.

(a) Outer layer

The outer layer generally comprises a matrix and a plurality of pharmaceutically active agents that are typically embedded in the matrix. Optionally, the outer layer may also include additional excipients.

### (i) Matrix

The matrix may be constructed from a variety of suitable excipients or combinations of excipients. Suitable excipients typically will yield a matrix that is substantially erodable when contacted with an aqueous medium, which includes any medium having water, such as saliva or a liquid containing water. In this context, “substantially erodable” means that the matrix typically will dissolve, disintegrate, or disperse when contacted with an aqueous medium and concomitantly, will then generally release the embedded pharmaceutically active agent or encapsulated pharmaceutically active agent. Without being bound to any particular limitation, the matrix will typically substantially erode within from about one second to about five minutes after being contacted with an aqueous medium.

The matrix may substantially be comprised of a carbohydrate. Suitable carbohydrates, in addition to being substantially erodable when contacted with an aqueous medium, include materials that may be embedded with a desired pharmaceutically active agent or a pharmaceutically active agent that has been encapsulated. The carbohydrate may be a polyol, low molecular weight saccharide, or any of the matrix materials, such as OraQuick Matrix, described in U.S. Pat. Nos. 6,284,270 and 6,465,010, both of which are hereby incorporated by reference in their entirety. Non-limiting examples of suitable carbohydrates include, mannitol, mannose, sorbitol, xylitol, xylose, dextrose, sucrose, lactose, glucose, fructose, and combinations thereof.

The amount of carbohydrate or other materials utilized to form the matrix can and will vary. For example, the matrix may comprise from about 1% to about 99% by weight of the outer layer. More typically, the matrix may comprise from about 60% to about 90% by weight of the outer layer.

As will be appreciated by a skilled artisan, depending upon the embodiment, the matrix may be in the form of either a continuous phase or in a discontinuous phase. Examples of matrices in a continuous phase include glassy, amorphous, and monocrystalline solids and semisolids. Alternatively, examples of a matrix in a discontinuous phase include singe phase particles bound to each other by e.g., either crystalline bridges and/or by other physical forces (e.g., Van der Waals or electrostatic).

(ii) Excipients

The outer layer may include one or more suitable excipients in addition to the matrix. Non-limiting examples of suitable excipients include an agent selected from the group consisting of non-effervescent disintegrants, a coloring agent, a flavor-modifying agent, an oral dispersing agent, a stabilizer, a preservative, a diluent, a compaction agent, a lubricant, a filler, a binder, and an effervescent disintegration agent. As will be appreciated by a skilled artisan, the combination of particular excipients utilized to form the outer layer can and preferably will vary depending upon a variety of parameters including the type of matrix and pharmaceutically active agent. The amount and types of excipients utilized to form the outer layer may be selected according to known principles of pharmaceutical science.

In one embodiment, the excipient is a binder. Suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂ -C₁₈ fatty acid alcohol, polyethylene glycol, polyols, saccharides, oligosaccharides, polypeptides, oligopeptides, and combinations thereof. The polypeptide may be any arrangement of amino acids ranging from about 100 to about 300,000 daltons. The amount of binding agent, if present, useful in the practice of the present invention may range from about 0.1% to about 25% by weight of the outer layer. More typically, the amount of binding agent may range from about 0.1% to about 10% by weight of the outer layer.

In another embodiment, the excipient may be a filler. Suitable fillers include carbohydrates, inorganic compounds, and polyvinilpirrolydone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, and sorbitol. The amount of filler may range from about 0.1% to about 75% by weight of the outer layer. More typically, the amount of filler may range from about 0.1% to about 25% by weight of the outer layer.

The excipient may comprise a non-effervescent disintegrant. Suitable examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-effervescent disintegrants may be present in an amount from about 2% to about 10% by weight of the outer layer.

In another embodiment, the excipient may be an effervescent disintegrant. By way of non-limiting example, suitable effervescent disintegrants include sodium bicarbonate in combination with citric acid and sodium bicarbonate in combination with tartaric acid. The effervescent disintegrants may be present in an amount from about 2% and about 10% by weight of the outer layer.

The excipient may comprise a preservative. Suitable examples of preservatives include antioxidants, such as a-tocopherol or ascorbate, and antimicrobials, such as parabens, chlorobutanol or phenol. The preservative is generally present in an amount of from about 0.001% to about 0.3% by weight of the outer layer.

In another embodiment, the excipient may include a diluent. Diluents suitable for use include pharmaceutically acceptable saccharide such as sucrose, dextrose, lactose, microcrystalline cellulose, fructose, xylitol, and sorbitol; polyhydric alcohols; a starch; pre-manufactured direct compression diluents; and mixtures of any of the foregoing. The diluents are generally present in an amount of from about 1% to about 10% by weight of the outer layer.

The excipient may include flavors. Flavors incorporated into the outer layer may be chosen from synthetic flavor oils and flavoring aromatics and/or natural oils, extracts from plants, leaves, flowers, fruits, and combinations thereof. By way of example, these may include cinnamon oils, oil of wintergreen, peppermint oils, clover oil, hay oil, anise oil, eucalyptus, vanilla, citrus oil, such as lemon oil, orange oil, grape and grapefruit oil, fruit essences including apple, peach, pear, strawberry, raspberry, cherry, plum, pineapple, and apricot. Typically, flavors may be present in an amount ranging from about 0.001% to 3.0% by weight of the outer layer.

In another embodiment, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof. Sweeteners may be present in an amount ranging from about 0.001% to 3.0% by weight of the outer layer.

In another embodiment, the excipient may be a lubricant. Suitable non-limiting examples of lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricant may be used in an amount ranging from about 0.001% to about 4% by weight of the outer layer.

The excipient may be a dispersion enhancer. Suitable dispersants may include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants. The dispersion enhancer may be used in an amount ranging from about 1% to about 10% by weight of the outer layer.

Depending upon the embodiment, it may be desirable to provide a coloring agent in the outer layer. Suitable color additives include food, drug and cosmetic colors (FD&C), drug and cosmetic colors (D&C), or external drug and cosmetic colors (Ext. D&C). These colors or dyes, along with their corresponding lakes, and certain natural and derived colorants may be suitable for use in the present invention depending on the embodiment. Generally speaking, the coloring agent may be present in an amount ranging from about 0.1% to 3.5% by weight of the outer layer.

(iii) Pharmaceutically Active Agent

The outer layer may be formulated to include any desired pharmaceutically active agent useful in the practice of the present invention. In certain embodiments, the pharmaceutically active agent may be encapsulated by a coating. Irrespective of whether the pharmaceutically active is encapsulated, it may comprise systemically distributable pharmaceutical ingredients such as, vitamins, minerals, and dietary supplements. Alternatively, the pharmaceutically active agent may include non-systemically distributable drugs. The pharmaceutically active agent may also include combinations of two, three, or four or more systemically distributable pharmaceutical ingredients or non-systemically distributable drugs.

Suitable pharmaceutically active agents, include, without limitation, an opioid analgesic agent (e.g., as morphine, hydromorphone, oxymorphone, levophanol, methadone, meperidine, fentanyl, codeine, hydrocodone, oxycodone, propoxyphene, buprenorphine, butorphanol, pentazocine and nalbuphine); a non-opioid analgesic agent (e.g., acetylsalicylic acid, acetaminophen, ibuprofen, ketoprofen, indomethacin, diflunisol, naproxen, ketorolac, dichlophenac, tolmetin, sulindac, phenacetin, piroxicam, and mefamanic acid); an anti-inflammatory agent (e.g., glucocorticoids such as alclometasone, fluocinonide, methylprednisolone, triamcinolone and dexamethasone; and non-steroidal anti-inflammatory drugs such as celecoxib, deracoxib, ketoprofen, lumiracoxib, meloxicam, parecoxib, rofecoxib, and valdecoxib); an antitussive agent (e.g., dextromethorphan, codeine, hydrocodone, caramiphen, carbetapentane, and dextromethorphan); an antipyretic agent (e.g., acetylsalicylic acid and acetaminophen); an antibiotic agent (e.g., aminoglycosides such as, amikacin, gentamicin, kanamycin, neomycin, netilmicin, streptorycin, and tobramycin; carbecephem such as loracarbef; carbapenems such as certapenem, imipenem, and meropenem; cephalosporins such as cefadroxil cefazolin, cephalexin, cefaclor, cefamandole, cephalexin, cefoxitin, cetprozil, cefuroxime, cetftazidime, cefdinir, cefditoren, cetoperazone, cefotaxime, cetpodoxime, ceftazidime, ceftibuten, ceftizoxime, and ceftriaxone; macrolides such as azithromycin, clarithromycin, dirithromycin, erythromycin and troleandomycin; monobactam, penicillins such as amoxicillin, ampicillin, carbenicillin, cloxacillin, dicioxacillin, nafilcillin, oxacillin, penicilin G, penicillin V, piperacillin, and ticarcillin; polypeptides such as bacitracin colstin, and polymycin B; quinolones such as ciprofloxacin, enoxacin, gatifloxacin, levofloxacin, ometloxacin, moxfolxacin, norfloxacin, ofloxacin and trovatioxacin, sulfonamides such as mafenide sulfacetamide, sulfamethizole, sulfasalazine, sulfisoxazole, and trimethoprim-sulfamethoxazole; and tetracyclines such as demeclocycline, doxycycline, minocycine, and oxytetracycline); an antimicrobial agent (e.g., ketoconazole, amoxicillin, cephalexin, miconazole, econazole, acyclovir, and nelfinavir); a steroidal agent (e.g., estradiol, testosterone, cortisol, aldosterone, prednisone, and cortisone); an amphetamine stimulant agent (e.g., amphetamine); a non-amphetamine stimulant agent (e.g., methylphenidate, nicotine, and caffeine); a laxative agent (e.g., bisacodyl, casanthranol, senna, and castor oil); an anorexic agent (e.g., fenfluramine, dexfenfluramine, mazindol, phentermine, and aminorex); an antihistaminic agent (e.g., phencarol, cetirizine, cinnarizine, ethamidindole, azatadine, brompheniramine, hydroxyzine, and chlorpheniramine); an antiasthmatic agent (e.g., zileuton, montelukast, omalizumab, fluticasone, and zafirlukast); an antidiuretic agent (e.g., desmopressin, vasopressin, and lypressin); an antiflatulant agent (e.g., simethicone); an antimigraine agent (e.g., naratriptan, frovatriptan, eletriptan, dihydroergotamine, zolmitriptan, almotriptan, and sumatriptan); an antispasmodic agent (e.g., dicyclomine, hyoscyamine, and peppermint oil); an antidiabetic agent (e.g., methformin, acarbose, miglitol, pioglitazone, rosiglitazone, troglitazone, nateglinide, repaglinide, mitiglinide, saxagliptin, sitagliptine, vildagliptin, acetohexamide, chlorpropamide, gliclazide, glimepiride, glipizide, glyburide, tolazamide, and tolbutamide); an antacid (e.g., aluminium hydroxide, magnesium hydroxide, calcium carbonate, sodium bicarbonate, and bismuth subsalicylate); a respiratory agent (e.g., albuterol, ephedrine, metaproterenol, and terbutaline); a sympathomimetic agent (e.g., pseudoephedrine, phenylephrine, phenylpropanolamine, epinephrine, norepinephrine, dopamine, and ephedrine); an H₂ blocking agent (e.g., cimetidine, famotidine, nizatidine, and ranitidine); an antihyperlipidemic agent (e.g., clofibrate, cholestyramine, colestipol, fluvastatin, atorvastatin, genfibrozil, lovastatin, niacin, pravastatin, fenofibrate, colesevelam, and simvastatin); an antihypercholesterol agent (e.g., lovastatin, simvastatin, pravastatin, fluvastatin, atorvastatin, cholestyramine, colestipol, colesevelam, nicotinic acid, gemfibrozil, and ezetimibe); a cardiotonic agent (e.g., digitalis, ubidecarenone, and dopamine); a vasodilating agent (e.g., nitroglycerin, captopril, dihydralazine, diltiazem, and isosorbide dinitrate); a vasocontricting agent (e.g., dihydroergotoxine and dihydroergotamine); a sedative agent (e.g., amobarbital, pentobarbital, secobarbital, clomethiazole, diphenhydramine hydrochloride, and alprazolam); a hypnotic agent (e.g., zaleplon, zolpidem, eszopiclone, zopiclone, chloral hydrate, and clomethiazole); an anticonvulsant agent (e.g., lamitrogene, oxycarbamezine, pheytoin, mephenytoin, ethosuximide, methsuccimide, carbamazepine, valproic acid, gabapentin, topiramate, felbamate, and phenobarbital); a muscle relaxing agent (e.g., baclofen, carisoprodol, chlorzoxazone, cyclobenzaprine, dantrolene sodium, metaxalone, orphenadrine, pancuronium bromide, and tizanidine); an antipsychotic agent (e.g., phenothiazine, chlorpromazine, fluphenazine, perphenazine, prochlorperazine, thioridazine, trifluoperazine, haloperidol, droperidol, pimozide, clozapine, olanzapine, risperidone, quetiapine, ziprasidone, melperone, and paliperidone); an antianxiolitic agent (e.g., lorazepam, alprazolam, clonazepam, diazepam, buspirone, meprobamate, and flunitrazepam); an antihyperactive agent (e.g., methylphenidate, amphetamine, and dextroamphetamine); an antihypertensive agent (e.g., alpha-methyldopa, chlortalidone, reserpine, syrosingopine, rescinnamine, prazosin, phentolamine, felodipine, propanolol, pindolol, labetalol, clonidine, captopril, enalapril, and lisonopril); an anti-neoplasia agent (e.g., taxol, actinomycin, bleomycin A₂, mitomycin C, daunorubicin, doxorubicin, epirubicin, idarubicin, and mitoxantrone); a soporific agent (e.g., zolpidem tartrate, eszopiclone, ramelteon, and zaleplon); a tranquilizer (e.g., alprazolam, clonazepam, diazepam, flunitrazepam, lorazepam, triazolam, chlorpromazine, fluphenazine, haloperidol, loxapine succinate, perphenazine, prochlorperazine, thiothixene, and trifluoperazine); a decongestant (e.g., ephedrine, phenylephrine, naphazoline, and tetrahydrozoline); a beta blocker (e.g., levobunolol, pindolol, timolol maleate, bisoprolol, carvedilol, and butoxamine); an alpha blocker (e.g., doxazosin, prazosin, phenoxybenzamine, phentolamine, tamsulosin, alfuzosin, and terazosin); a non-steroidal hormone (e.g., corticotropin, vasopressin, oxytocin, insulin, oxendolone, thyroid hormone, and adrenal hormone); a herbal agent (e.g., glycyrrhiza, aloe, garlic, nigella sativa, rauwolfia, St John's wort, and valerian); an enzyme (e.g., lipase, protease, amylase, lactase, lysozyme, and urokinase); a humoral agent (e.g., prostaglandins, natural and synthetic, for example, PGE₁, PGE₂alpha, and PGF₂alpha, and the PGE₁ analog misoprostol); a psychic energizer (e.g., 3-(2-aminopropy)indole and 3-(2-aminobutyl)indole); a vitamin (e.g., retinol, retinal, retinoic acid, 3-dehydroretinol, thiamine, riboflavin, niacin, pantothenic acid, pyridoxine, biotin, folic acid, cyanocobalamin, ascorbic acid, lumisterol, ergocalciferol, cholecalciferol, dihydrotachysterol, tocopherol, and naphthoquinone); a mineral (e.g., calcium, iron, zinc, selenium, copper, iodine, magnesium, phosphorus, and chromium); an anti-nausea agent (e.g., dolasetron, granisetron, ondansetron, tropisetron, meclizine, and cyclizine); a hematinic agent (e.g., ferrous salts, ferrous amino chelates, ferrous sulfate, ferrous fumarate, Ferrochel iron); a nutritional product (e.g., bee pollen, bran, wheat germ, kelp, cod liver oil, ginseng, and fish oils, amino acids, proteins, and mixtures thereof); and a fiber product (e.g., cellulose, lignin, waxes, chitins, pectins, beta-glucans, inulin, and oligosaccharides).

The amount of pharmaceutically active agent (or microencapsulated pharmaceutically active agent) embedded in the outer layer may be selected according to known principles of pharmacy. Generally speaking, an “effective amount” of pharmaceutically active agent is specifically contemplated. When the term “effective amount” refers to pharmaceuticals, a pharmaceutically effective amount is contemplated. In this context, a pharmaceutically effective amount is the amount or quantity of a drug or pharmaceutically active agent that is sufficient to elicit the required or desired therapeutic response. Alternatively, when the term “effective amount” refers to a vitamin or mineral, it quantifies an amount at least about 10% of the United States Recommended Daily Allowance (“RDA”) of that particular ingredient, i.e., vitamin or mineral, for a subject. It is contemplated, however, that amounts of certain minerals or vitamins exceeding the RDA may be beneficial for certain subjects. For example, the amount of a given vitamin or mineral may exceed the applicable RDA by 100%, 200%, 300%, 400% or 500% or more.

As will be appreciated by a skilled artisan, the amount of pharmaceutically active agent embedded in the matrix can vary widely from 0.01 micrograms to 10 grams or more. More typically, the amount will range from a few micrograms to several milligrams or more.

(iv) Microcapsules

To provide a mechanism to mask the taste of the pharmaceutically active agent or to provide a means for its controlled release, another aspect of the invention provides a microcapsule comprising a pharmaceutically active agent and a coating that encapsulates the pharmaceutically active agent. In this aspect of the invention, as such, the outer layer comprises a plurality of microcapsules embedded in the matrix. The microcapsule will generally comprise any of the pharmaceutically active agents, or combinations of any of the pharmaceutically active agents as detailed in (I)(a)(iii) encapsulated by a coating.

The coating can and will vary depending upon a variety of factors, including, the pharmaceutically active agent, and the purpose to be achieved by its encapsulation (e.g., flavor masking, maintenance of structural integrity, or formulation for time release). The coating material may be a biopolymer, a semi-synthetic polymer, or a mixture thereof. The microcapsule may comprise one coating layer or many coating layers, of which the layers may be of the same material or different materials. In one embodiment, the coating material may comprise a polysaccharide or a mixture of saccharides and glycoproteins extracted from a plant, fungus, or microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In another embodiment, the coating material may comprise a protein. Suitable proteins include, but are not limited to, gelatin, casein, collagen, whey proteins, soy proteins, rice protein, and corn proteins. In an alternate embodiment, the coating material may comprise a fat or oil, and in particular, a high temperature melting fat or oil. The fat or oil may be hydrogenated or partially hydrogenated, and preferably is derived from a plant. The fat or oil may comprise glycerides, free fatty acids, fatty acid esters, or a mixture thereof. In still another embodiment, the coating material may comprise an edible wax. Edible waxes may be derived from animals, insects, or plants. Non-limiting examples include beeswax, lanolin, bayberry wax, carnauba wax, and rice bran wax. The coating material may also comprise a mixture of biopolymers. As an example, the coating material may comprise a mixture of a polysaccharide and a fat.

In an exemplary embodiment, the coating may be an enteric coating. The enteric coating generally will provide for controlled release of the pharmaceutically active agent, such that drug release can be accomplished at some generally predictable location in the lower intestinal tract below the point at which drug release would occur without the enteric coating. In certain embodiments, multiple enteric coatings may be utilized. Multiple enteric coatings, in certain embodiments, may be selected to release the pharmaceutically active agent at various regions in the lower gastrointestinal tract.

The enteric coating is typically, although not necessarily, a polymeric material that is pH sensitive. A variety of anionic polymers exhibiting a pH-dependent solubility profile may be suitably used as an enteric coating in the practice of the present invention to achieve delivery of the active to the lower gastrointestinal tract. Suitable enteric coating materials include, but are not limited to: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). Combinations of different coating materials may also be used to coat a single capsule.

The thickness of a microcapsule coating may be an important factor in some instances. For example, the “coating weight,” or relative amount of coating material per dosage form, generally dictates the time interval between oral ingestion and drug release. As such, a coating utilized for time release of the pharmaceutically active agent into the gastrointestinal tract is typically applied to a sufficient thickness such that the entire coating does not dissolve in the gastrointestinal fluids at pH below about 5, but does dissolve at pH about 5 and above.

The thickness of a microcapsule coating of the present invention may be expressed as a percentage representing the ratio of the volume of the coating to the weight of the pharmaceutically active agent. Accordingly, the volume ratio of coating to pharmaceutically active agent may be less than about 95% (e.g., between about 1% or 5% and about 95%). Alternatively, the volume ratio may be less than about 75% (e.g., between about 1% and 75%). In still another embodiment, the volume ratio is less than about 65% (e.g., between about 5% and 65%). Generally then, for microcapsules having a coating to pharmaceutically active agent volume ratio between about 12% and about 70%, the equivalent thickness of coating is between about 5% and about 30% of the diameter of a microcapsule.

The size and shape of the microcapsules can and will vary without departing from the scope of the present invention. For example, the microcapsules may be spherical, regularly shaped, irregular, or combinations thereof. Generally, their size may be measured in terms of the diameter of a sphere that occupies the same volume as the microcapsule being measured. The characteristic diameter of a microcapsule may be directly determined, for example, by inspection of a photomicrograph. The size of the microcapsules can and will vary, depending upon the condition used to form the particles and the type of encapsulation. Typically, microcapsules of the present invention may have an average diameter from a few nanometers to about 200 microns. In an exemplary embodiment, the microcapsules will have an average diameter less than about 200 microns, less than about 150 microns, less than about 100 microns, less than about 75 microns, less than about 50 microns, or less than about 25 microns.

As will be appreciated by a skilled artisan, the encapsulation method can and will vary depending upon the compounds used to form the pharmaceutically active agent and coating, and the desired physical characteristics of the microcapsules themselves. Additionally, more than one encapsulation method may be employed so as to create a multi-layered microcapsule, or the same encapsulation method may be employed sequentially so as to create a multi-layered microcapsule. Suitable methods of microencapsulation may include spray drying, spinning disk encapsulation (also known as rotational suspension separation encapsulation), supercritical fluid encapsulation, air suspension microencapsulation, fluidized bed encapsulation, spray cooling/chilling (including matrix encapsulation), extrusion encapsulation, centrifugal extrusion, coacervation, alginate beads, liposome encapsulation, inclusion encapsulation, colloidosome encapsulation, sol-gel microencapsulation, and other methods of microencapsulation known in the art. Detailed information concerning materials, equipment and processes for preparing coated dosage forms may be found in Pharmaceutical Dosage Forms: Tablets, eds. Lieberman et al. (New York: Marcel Dekker, Inc., 1989), and in Ansel et al., Pharmaceutical Dosage Forms and Drug Delivery Systems, 6^{th} Ed. (Media, Pa.: Williams & Wilkins, 1995).

(v) Formation of the Outer Layer

The process utilized to construct the outer layer can and preferably will vary. By way of non-limiting example, the desired amount of matrix, excipients, and pharmaceutically active agents may be combined and mixed with water. The mixture may then be granulated in a high shear granulator, the granulation may be dried, and the outer layer composition may then be screened to produce particles having a desired size. Other process known in the art may be utilized to form the outer layer, including the processes detailed in U.S. Pat. Nos. 6,248,279 and 6,465,010, both of which are hereby incorporated by reference in their entirety.

Generally speaking, the outer layer will typically comprise the pharmaceutically active agent in an amount from about 0.001% to about 95% by weight of the outer layer, and the matrix in an amount from about 1% to about 99% by weight of the outer layer. More typically, the outer layer will comprise the pharmaceutically active agent in an amount from about 0.1% to about 30% by weight of the outer layer, and the matrix in an amount from about 60% to about 90% by weight of the outer layer. In each embodiment, the outer layer may comprise a binder in an amount from about 0.1% to about 25% by weight of the outer layer and a filler in an amount from about 0.1% to about 75% by weight of the outer layer. More typically, the outer layer may comprise a binder in an amount from about 0.1% to about 10% by weight of the outer layer and a filler in an amount from about 0.1% to about 25% by weight of the outer layer.

For embodiments where the outer layer is formed over an inner core, it may be applied by methods generally known in the art, such as by dry powder layering or by a fluid bed process. Detailed information concerning materials, equipment and processes for preparing and applying an outer layer over in inner core may be found in Pharmaceutical Dosage Forms: Tablets, eds. Lieberman et al. (New York: Marcel Dekker, Inc., 1989), and in Ansel et al., Pharmaceutical Dosage Forms and Drug Delivery Systems, 6^{th} Ed. (Media, Pa.: Williams & Wilkins, 1995).

### (b) Inner Core

The inner core, if present, is generally coated with the outer layer comprising the matrix and plurality of pharmaceutically active agents or encapsulated pharmaceutically active agents. Without being bound to any particular theory, the inner core is desirable in certain embodiments to provide greater uniformity for drug delivery, and to provide more surface area to which the outer layer may be applied. In this context, the inner core provides a means to increase the amount of pharmaceutically active agent that may be added to the oral delivery compositions of the invention while maintaining uniform dosage forms. The inner core comprises an inert material, and may optionally include an indicator coating. In certain embodiments, the inner core is substantially soluble such that is dissolves when contacted with an aqueous medium. In other embodiments, the inner core is substantially insoluble such that it does not dissolve when contacted with an aqueous medium.

(i) Inert Material

The inert material forming the inner core may comprise a variety of suitable materials to the extent they are substantially non reactive with other materials forming the composition of the invention, and in particular, the pharmaceutically active layer. In some embodiments, the suitable materials include without limitation nonpareil sugar beads, tapioca starch beads, complex alumosilcate granules, activated charcoal granules, and sugar compositions. In an exemplary embodiment, the inert material comprises a sugar composition.

Generally speaking, the size of the inner core can and will vary. The inner core may range from about 50 microns to approximately 500 times greater by weight than the weight of the pharmaceutically active agent. In other embodiments, the inner core may be from about 5 times, 10 times, 15 times, 20 times, 25 times, 30 times, 35 times, 40 times, 45 times, 50 times, 55 times, 60 times, 65 times, 70 times, 75 times, 80 times, 90 times, 95 times, 100 times or greater than 250 times greater by weight than the weight of the pharmaceutically active agent. Stated another way, weight ratio of the inner core to the outer layer may be from about 0% to about 10%, from about 5% to about 25%, from about 20% to about 60%, or from about 50% to about 75% by weight of the outer layer. For embodiments where the oral delivery composition is included in a device where liquid passes through (e.g., a drinking straw), if the inner core is substantially insoluble it is typically larger than the aperture of the device.

In another embodiment, the inner core may substantially dissolve when contacted with an aqueous medium. In one alternative of this embodiment, the inner core may dissolve after the matrix has substantially eroded. Alternatively, the inner core may dissolve approximately simultaneously as the matrix erodes.

(ii) Indicator Coating

As an aide to determine when the desired amount of pharmaceutically active agent has been administered to the subject, an indicator coating, such as a color indicator, may be formed over the inner core (i.e., disposed between the inner core and outer layer). The mechanism of detection utilized in the indicator system can and will vary. The mechanism may be based on smell, sight, taste, or touch. In an exemplary embodiment, a color indicator detectable by sight is utilized. By way of non-limiting example, when the indicator coating comprises acryl-Eze red (as illustrated in the examples) the coating becomes visible as the pharmaceutically active agent is released from the matrix when the matrix is contacted with an aqueous medium. In another embodiment, the inner core may be colored throughout. Suitable color indicators are generally substantially insoluble when contacted with an aqueous medium and include acryl-Eze, lakes, and other food and FD&C dyes. By way of further non-limiting example, the inner core may be substantially soluble when contacted with an aqueous medium after the matrix erodes. For this embodiment, the inner core may release a flavor into the aqueous medium to indicate when the pharmaceutically active agent has been released. Alternatively, the inner core may release a color, which changes the color of the aqueous medium to indicate when the pharmaceutically active agent has been released.

An inner core coated with an indicator coating will generally comprise an inert material in an amount from about 70% to about 90% by weight of the inner core and an indicator coating in an amount from about 10% to about 30% by weight of the inner core. In another embodiment, the inner core coated with an indicator coating will comprise an inert material in an amount from about 75% to about 99% and an indicator coating in an amount from about 1% to about 10% by weight of the inner core. More typically, the inner core coated with an indicator coating will comprise an inert material in an amount from about 80% to about 90% by weight of the inner core and an indicator coating in an amount from about 10% to about 20% by weight of the inner core. The indicator coating may be applied to the inner core by any of the methods detailed herein, such as in the examples or as detailed in (I)(a)(v), or as otherwise known in the art.

### (II) Method for Oral Delivery

Another aspect of the invention provides a method for orally delivering a pharmaceutically active agent to a subject by introducing a composition of the invention into the subject's oral cavity. As detailed above, when the compositions are contacted with an aqueous medium, such as saliva or water, the matrix erodes and releases the pharmaceutically active agents.

The oral delivery composition may be introduced into the oral cavity of a subject by any of a variety of methods known in the art for oral drug, vitamin, or mineral delivery. For example, the oral delivery composition may be formulated into a conventional tablet or pill. More typically, however, the composition will be introduced into the subject's oral cavity as a pellet, bead, powder, sachet, soft chew, hard candy, or sprinkle.

In an exemplary embodiment, the composition will be combined with an aqueous-based liquid prior to its introduction into the subject's oral cavity. Non-limiting examples of a liquid beverage suitable for use include milk, flavored milk drinks, goat milk, liquid yogurt, soy milk, rice milk, fruit drinks, fruit-flavored drinks, vegetable drinks, nutritional drinks, energy drinks, sports drinks, infant formula, teas, and coffee drinks. Generally for this embodiment, the subject consumes the liquid through a drinking straw or other similar device. The inside of the drinking straw may either be coated with the composition of the invention or it may contain the composition in bead form. When the straw contains beads comprising the composition, the straw also generally has a porous material disposed on both of its ends. Typically, the porous material prevents the beads from leaving the straw, but allows fluid to readily exit as the subject imbibes the liquid beverage, or prevents the beads from leaving the straw in a size that would not be tolerated or safe if consumed by the subject. As the liquid beverage passes through the straw and is contacted with the beads, the matrix erodes and releases the pharmaceutically active agent, which is administered to the subject as the beverage is consumed. The inner core and portions of the matrix typically remain in the straw. Alternatively, in other embodiments the inner core and portions of the matrix may substantially dissolve and be consumed by the subject together with the liquid beverage. In an exemplary embodiment, the beads utilized in the straw will comprise an inner core with an indicator coating. The indicator coating visually changes color, such as from black to red, or the indicator color of the core is exposed as the pharmaceutically active agent is released from the matrix, thus indicating when the pharmaceutically active agent has been dispensed to the subject.

In another exemplary embodiment, the composition may be in the form of a powder, pellet, bead, soft chew, or sprinkle and introduced into the subject's oral via a food product. For example, the composition may be added to the outside of the food product after the food product's manufacture or it may be combined with the ingredients comprising the food product as it is manufactured. Typically, suitable food products include products that do not provide a substantial aqueous medium. In an embodiment, the food product may be a dry food (e.g., cereal-based product) that when milk or any aqueous liquid is added, results in the matrix substantially eroding releasing the pharmaceutically active agent in an aqueous phase and optionally, may also result in the inner core eroding (which may result in the release of a color or flavor indicator). Non-limiting examples of food products derived from cereal include breakfast cereals, pasta, breads, baked products (i.e., cakes, pies, rolls, cookies, crackers), tortillas, granola bars, nutrition bars, and energy bars. The food product may be a nutritional supplement. In still another embodiment, the food product may be a vegetable-derived product. Examples of vegetable-derived food products include textured vegetable proteins, tofu, corn chips, potato chips, vegetable chips, popcorn, and chocolate products. The food product may be a chewing gum. In certain embodiments for food and chewable applications, the composition may comprise an inner core that substantially dissolves when contacted with an aqueous medium. In other embodiments, the inner core may remain substantially intact when contacted with an aqueous medium.

As will be appreciated by a skilled artisan, the oral composition may be beneficially utilized to administer a pharmaceutically active agent to a wide range of subjects including animals and humans. The animal may be an agricultural animal. Suitable examples include, but are not limited to, chicken, beef cattle, dairy cattle, swine, sheep, goat, horse, duck, turkey, and goose. The animal may be a companion animal, such as cat, rabbit, rat, hamster, parrot, horse, or dog. The animal may also be an aquatic animal, such as fish or shellfish. Alternatively, the animal may be a game animal or a wild animal. Non-limiting examples of suitable game animals include buffalo, deer, elk, moose, reindeer, caribou, antelope, rabbit, squirrel, beaver, muskrat, opossum, raccoon, armadillo, porcupine, pheasant quail, and snake. In an exemplary embodiment, the subject is a human. In a particularly preferred embodiment, the subject is a human that has difficulty swallowing. In one alternative of this embodiment, the subject is a child. In another alternative of this embodiment, the subject is elderly. In a further embodiment, the subject has an illness, such as cancer, Acquired Immune Deficiency Syndrome (AIDS), the flu, pneumonia and other similar illnesses resulting in weakness or a difficulty in swallowing or consuming adequate nutrition.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

The following examples illustrate various iterations of the invention.

### Example 1

### Process for Making an Oral Delivery Composition with No Inner Core

Composition:

| Ingredient | Amount per batch | % of formula |
|---|---|---|
| MicroMask Phenylephrine | 20 g | 4.0% |
| Hydrochloride, 56% | | |
| Microcrystalline Cellulose | 137 g | 27.4% |
| Spray Dried Mannitol | 343 g | 68.6% |

The above components were mixed and granulated in a high shear granulator in the presence of water. The resulting granulation slurry was dried in a tray drier for 12 hours and passed through a number 16 mesh US Standard screen. Using this process, pellets containing 20 mg/g Phenylephrine Hydrochloride were produced.

### Example 2

### Process for Making an Inner Core

Composition:

% of formula

| | Amount per | |
|---|---|---|
| Ingredient | batch | % of formula |
| Sugar Spheres 1418, NF | 800 g | 80% |
| Acryl-Eze, Red (Colorcon) | 200 g | 20% |

Sugar Spheres, NF were coated with Acryl-Eze, Red (Colorcon) in a fluid bed Wurster column to 20% weight gain in accordance with generally known methods to form an inner core. The resulting beads are characterized by dark red color.

The composition produced in Example 1 may be applied to the inner core via dry powder layering, which uses a fluid bed apparatus with a rotary attachment (Glatt or Vector), or a stand-alone rotary granulator (Granurex® manufactured by Vector Corp.). Alternatively a fluid bed process may be performed using a Wurster column or a conventional top or tangential spray configuration.

The red inner core particles will become visible upon release of the pharmaceutically active agent from the matrix.

### Example 3

### Process for Making an Oral Delivery Composition with an Encapsulated Pharmaceutically Active Agent

A pellet with microencapsulated Phenylephrine Hydrochloride Red core beads, lot PDDI80 (as set forth in Example 2) were loaded into a rotary granulator processor of a GPCG-1 multifunction fluid bed apparatus (Glatt Air Techniques). A dry powder mixture of the composition made in Example 1 containing encapsulated Phenylephrine Hydrochloride (MicroMask® Phenylephrine HCl, 56%) was layered onto the Red Core Beads while using 5% aqueous solution of polyvinyl pyrrolidone (Povidone K90, ISP Inc.) as binder. Approximately 2.5 to 2.8 mm diameter beads were produced. The beads (Lot GV060046) are characterized by the following composition.

SDS Phenylephrine HCl (GV060046) Bead composition:

| Ingredient | Amount in formula |
|---|---|
| MicroMask ® Phenylephrine Hydrochloride 56% | 3.2% |
| Microcrystalline Cellulose, PH 102 (IFP) | 6.6% |
| Citric Acid | 2.4% |
| Povidone K90 | 8.0% |
| Red sugar core | 10.3% |
| Spray Dried Mannitol | 60.1% |
| Pray Dried Sorbitol | 7.4% |

As another example, an oral delivery composition with microencapsulated dextromethorphan hydrobromide was made. In this the manufacture, Red core beads, lot PDDI80 (as set forth in Example 2) were loaded into the rotary granulator processor of a GPCG-1 multifunction fluid bed apparatus (Glatt Air Techniques). Dry powder mixture of a composition containing microencapsulated dextromethorphan hydrobromide (MicroMask dextromethorphan Her, 47%) was layered onto the Red Core Beads while using 5% aqueous solution of polyvinyl pyrrolidone (Povidone K90, ISP Inc.) as binder. Approximately 2.8 to 3.0 mm diameter beads were produced. The beads (Lot GV060047) are characterized by the following composition

SDS Dextromethorphan HBr (GV060047) Bead Composition

| Ingredient | Amount in formula |
|---|---|
| MicroMask Dextromethorphan, 47% | 8.7% |
| Spray Dried Mannitol | 15.3% |
| Microcrystalline Cellulose, PH 102 (IFP) | 1.9% |
| Citric Acid | 1.9% |
| Povidone K90 | 6.3% |
| Red sugar cores | 8.6% |
| F-Melt, type M (Fuji Health Science) | 56.3% |

As another example, any of the oral delivery pharmaceutical compositions detailed in examples 1 through 3 may include a nutrient such as a vitamin or mineral in lieu of the pharmaceutical agent. In this context, the vitamin or mineral may be administered to a subject via a straw, powder, soft chew, hard candy or sprinkle.

While there is shown and described herein certain compositions for oral delivery of pharmaceuticals in accordance with the present invention, it will be manifest to those skilled in the art that various modifications may be made without departing from the spirit and scope of the underlying inventive concept and that the same is not limited to particular compositions herein shown and described except insofar as indicated by the scope of the appended claims.

What is claimed is:

1. A composition comprising an outer layer formed over an inner core, the inner core comprising an inert material, the outer layer comprising a plurality of pharmaceutically active agents embedded in a matrix, the matrix being substantially erodable when contacted with an aqueous medium.

2. The composition of claim 1, wherein the matrix comprises a carbohydrate.

3. The composition of claim 1, wherein the matrix substantially erodes within from about 1 second to about 5 minutes after being contacted with an aqueous medium.

4. The composition of claim 1, wherein the inert material comprises a sugar composition.

5. The composition of claim 1, wherein the pharmaceutically active agent is at least one ingredient selected from the group consisting of an opioid analgesic agent, a non-opioid analgesic agent, an anti-inflammatory agent, an antitussive agent, an antipyretic agent, an antibiotic agent, an antimicrobial agent, a steroidal agent, an amphetamine stimulant agent, a non-amphetamine stimulant agent, a laxative agent, an anorexic agent, an antihistaminic agent, an antiasthmatic agent, an antidiuretic agent, an antiflatulant agent, an antimigraine agent, an antispasmodic agent, an antidiabetic agent, an antacid, a respiratory agent, a sympathomimetic agent, an H₂ blocking agent, an antihyperlipidemic agent, an antihypercholesterol agent, a cardiotonic agent, a vasodilating agent, a vasocontricting agent, a sedative agent, a hypnotic agent, an anticonvulsant agent, a muscle relaxing agent, an antipsychotic agent, an antianxiolitic agent, an antihyperactive agent, an antihypertensive agent, an anti-neoplasia agent, a soporific agent, a tranquilizer, a decongestant, a beta blocker, an alpha blocker, a non-steroidal hormone, a herbal agent, an enzyme, a humoral agent, a psychic energizer, a vitamin, a mineral, an anti-nausea agent, a hematinic agent, a nutritional product, and a fiber product.

6. The composition of claim 1, further comprising an indicator coating formed over the inner core and disposed between the inner core and the outer layer.

7. The composition of claim 6, wherein the indicator coating may or may not change color when most of the pharmaceutically active agent is released from the matrix.

8. The composition of claim 6, wherein the indicator coating is substantially insoluble when contacted with an aqueous medium.

9. The composition of claim 1, wherein the outer layer further comprises a binder and/or a filler.

10. The composition of claim 1, wherein the outer layer comprises the pharmaceutically active agent in an amount from about 0.001% to about 95% by weight of the outer layer, and the matrix in an amount from about 1% to about 99% by weight of the outer layer; and the inner core is from about 1% to about 75% by weight of the outer layer.

11. The composition of claim 10, wherein the outer layer further comprises a binder in an amount from about 0.1% to about 25% by weight of the outer layer and a filler in an amount from about 0.1% to about 75% by weight of the outer layer.

12. The composition of claim 11, wherein the outer layer comprises the pharmaceutically active agent in an amount from about 0.1% to about 30% by weight of the outer layer, the matrix in an amount from about 60% to about 90% by weight of the outer layer, the binder in an amount from about 0.1% to about 10% by weight of the outer layer, and the filler in an amount from about 0.1% to about 25% by weight of the outer layer.

13. The composition of claim 6, wherein the indicator coated inner core comprises a carbohydrate composition in an amount from about 70% to about 99% by weight of the indicator coated inner core and an indicator coating in an amount from about 1% to about 30% by weight of the indicator coated inner core.

14. The composition of claim 13, wherein the indicator coated inner core comprises a sugar composition in an amount of about 80% by weight of the indicator coated inner core and an indicator coating in an amount of about 20% by weight of the color coated inner core.

15. The composition of claim 13, wherein the outer layer comprises the pharmaceutically active agent in an amount from about 0.1% to about 99% by weight of the outer layer, the matrix in an amount from about 1% to about 90% by weight of the outer layer, the binder in an amount from about 0.1% to about 10% by weight of the outer layer, and the filler in an amount from about 0.1% to about 25% by weight of the outer layer.

16. The composition of claim 15, wherein the outer layer further comprises at least one excipient selected from the group consisting of non-effervescent disintegrants, a coloring agent, a flavor-modifying agent, an oral dispersing agent, a stabilizer, a preservative, a diluent, a compaction agent, a lubricant, a filler, a binder, and an effervescent disintegration agent.

17. The composition of claim 1, wherein the pharmaceutically active agents have an average diameter of less than about 200 microns.

18. The composition of claim 1, wherein the pharmaceutically active agent is present in an amount ranging from 0.01 micrograms to about 10 grams.

19. The composition of claim 1 wherein each pharmaceutically active agent is encapsulated in a microcapsule, each microcapsule comprising a pharmaceutically active agent and a coating that encapsulates the pharmaceutically active agent.

20. The composition of claim 19, wherein the coating encapsulating the pharmaceutically active agent is an enteric coating.

21. The composition of claim 20, wherein the enteric coating is selected from a pH sensitive coating, a lipid soluble coating, and a water-soluble coating.

22. The composition of claim 19, wherein the pharmaceutically active agent is at least one ingredient selected from the group consisting of an opioid analgesic agent, a non-opioid analgesic agent, an anti-inflammatory agent, an antitussive agent, an antipyretic agent, an antibiotic agent, an antimicrobial agent, a steroidal agent, an amphetamine stimulant agent, a non-amphetamine stimulant agent, a laxative agent, an anorexic agent, an antihistaminic agent, an antiasthmatic agent, an antidiuretic agent, an antiflatulant agent, an antimigraine agent, an antispasmodic agent, an antidiabetic agent, an antacid, a respiratory agent, a sympathomimetic agent, an H₂ blocking agent, an antihyperlipidemic agent, an antihypercholesterol agent, a cardiotonic agent, a vasodilating agent, a vasocontricting agent, a sedative agent, a hypnotic agent, an anticonvulsant agent, a muscle relaxing agent, an antipsychotic agent, an antianxiolitic agent, an antihyperactive agent, an antihypertensive agent, an anti-neoplasia agent, a soporific agent, a tranquilizer, a decongestant, a beta blocker, an alpha blocker, a non-steroidal hormone, a herbal agent, an enzyme, a humoral agent, a psychic energizer, a vitamin, a mineral, an anti-nausea agent, a hematinic agent, a nutritional product, and a fiber product.

23. The composition of claim 19, further comprising an indicator coating formed over the inner core and disposed between the inner core and the outer layer.

24. The composition of claim 23, wherein the indicator coating changes color when most of the pharmaceutically active agent is released from the matrix.

25. The composition of claim 19, wherein the indicator coating is substantially insoluble when contacted with an aqueous medium.

26. The composition of claim 19, wherein the outer layer further comprises a binder and/or a filler.

27. The composition of claim 19, wherein the outer layer comprises the pharmaceutically active agent in an amount from about 0.001% to about 95% by weight of the outer layer, and the matrix in an amount from about 1% to about 99% by weight of the outer layer; and the inner core is from about 1% to about 75% by weight of the outer layer.

28. The composition of claim 21, wherein the outer layer further comprises a binder in an amount from about 0.1% to about 25% by weight of the outer layer and a filler in an amount from about 0.1% to about 75% by weight of the outer layer.

29. The composition of claim 28, wherein the outer layer comprises the pharmaceutically active agent in an amount from about 0.1% to about 99% by weight of the outer layer, the matrix in an amount from about 1% to about 90% by weight of the outer layer, the binder in an amount from about 0.1% to about 10% by weight of the outer layer, and the filler in an amount from about 0.1% to about 25% by weight of the outer layer.

30. The composition of claim 23, wherein the indicator coated inner core comprises a carbohydrate composition in an amount from about 70% to about 99% by weight of the indicator coated inner core and an indicator coating in an amount from about 1% to about 30% by weight of the indicator coated inner core.

31. The composition of claim 30, wherein the indicator coated inner core comprises a sugar composition in an amount of about 80% by weight of the indicator coated inner core and a color coating in an amount of about 20% by weight of the indicator coated inner core.

32. The composition of claim 30, wherein the outer layer comprises the pharmaceutically active agent in an amount from about 0.1% to about 30% by weight of the outer layer, the matrix in an amount from about 60% to about 90% by weight of the outer layer, the binder in an amount from about 0.1% to about 10% by weight of the outer layer, and the filler in an amount from about 0.1% to about 25% by weight of the outer layer.

33. The composition of claim 32, wherein the outer layer further comprises at least one excipient selected from the group consisting of non-effervescent disintegrants, a coloring agent, a flavor-modifying agent, an oral dispersing agent, a stabilizer, a preservative, a diluent, a compaction agent, a lubricant, a filler, a binder, and an effervescent disintegration agent.

34. The composition of claim 19, wherein the microcapsules have an average diameter of less than about 200 microns.

35. The composition of claim 19, wherein the pharmaceutically active agent is present in an amount ranging from 0.01 micrograms to about 10 grams.

36. The composition of claim 19, wherein the microcapsules have an average diameter of from about 50 microns to less than approximately 250 microns.

37. A method for orally delivering a pharmaceutically active agent to a subject, the method comprising introducing a composition of claim 1 into the oral cavity of the subject.

38. The method of claim 37, further comprising combining the composition with an aqueous-based liquid prior to administering the composition to the subject.

39. The method of claim 37, wherein the subject is selected from the group consisting of a companion animal, an agricultural animal, and a human.

40. The method of claim 39, wherein the subject has difficulty swallowing.

41. The method of claim 39, wherein the subject is a human that is a child or elderly.

42. The method of claim 37, wherein the composition is introduced into the oral cavity of the subject by a means selected from the group consisting of a pellet, a powder, a sachet, a nutritional bar, a soft chew, a sprinkle, and a drinking straw.

43. The method of claim 37, wherein the composition is administered with a second pharmaceutically active agent.

44. The method of claim 43, wherein the composition and second pharmaceutically active agent are administered simultaneously or sequentially.

45. A method for manufacturing a composition for oral delivery of a pharmaceutical agent, the method comprising combining an inner core with an outer layer in a manner such that the outer layer is formed over the inner core, the inner core comprising an inert material, the outer layer comprising a plurality of pharmaceutically active agents embedded in a matrix, the matrix being substantially erodable when contacted with an aqueous medium.
